(19) United States
(12) Patent Application Publication
(10) Pub. No.: US 2013/0178790 A1
(43) Pub. Date: Jul. 11, 2013

(54) INFUSION CATHETER

(71) Applicant: Kurt J. Tekulve, Ellettsville, IN (US)

(72) Inventor: Kurt J. Tekulve, Ellettsville, IN (US)

(73) Assignees: Cook Medical Technologies LLC, Bloomington, IN (US); Cook Incorporated, Bloomington, IN (US)

(21) Appl. No.: 13/344,116

(22) Filed: Jan. 5, 2012

(51) Int. Cl. A61M 1/00; A61M 25/14

(52) U.S. Cl. 604/28; 604/35; 604/31

(57) ABSTRACT

The disclosure provides an apparatus and methods for removing thrombosis in vessels. The apparatus includes a sheath catheter combined with an aspiration catheter. The sheath catheter includes a first sheath opening in a proximal end of the sheath catheter, a second sheath opening in a distal end of the sheath catheter, a first balloon disposed on an outer surface of the distal end of the sheath catheter, and a passageway extending between the first and second sheath openings. The aspiration catheter is configured to pass through the first and second sheath openings and the passageway of the sheath catheter. The aspiration catheter includes a second balloon, a plurality of infusion ports, and an aspiration port disposed on an outer surface of a distal portion of the aspiration catheter. A relative position between the sheath catheter and the aspiration catheter controls the number of unblocked infusion ports.

### BACKGROUND

The present disclosure relates generally to a catheter device for removing thrombosis from vessels.

A blood clot may form in blood vessels and cause danger to a patient's life. For example, deep vein thrombosis (DVT) is the formation of a blood clot or thrombus in a deep vein. DVT may affect the leg veins such as the femoral vein or the popliteal vein or the deep veins of the pelvis. Occasionally, DVT may affect the veins of the arm. A DVT can occur without symptoms, but in many cases the affected extremity will be painful, swollen, red, and warm, and the superficial veins may be engorged. The most serious complication of a DVT is that the clot could dislodge and travel to the lungs, which is called a pulmonary embolism (PE). Thus, it is necessary to break up the clot and remove it as early as possible.

Catheters have been introduced as a minimally invasive device in treating DVT. One method is to use thrombolytics and an infusion catheter for the process. Another method is to use a filter and spin the filter to break up the clot and then remove the broken clot fragments. However, these methods may need a high dosage of thrombolytics or cannot guarantee removing all the broken clot fragments. Further, conventional catheters require the physician to use a sheath to introduce a catheter. Thus, the procedure is complicated and time consuming.

To solve these problems, the instant application provides a catheter device to remove the clot efficiently and completely.

### SUMMARY

The present disclosure provides a catheter apparatus for removing thrombosis.

One embodiment of the catheter apparatus comprises a sheath catheter combined with an aspiration catheter. The sheath catheter includes a first sheath opening in a proximal end of the sheath catheter, a second sheath opening in a distal end of the sheath catheter, a first balloon disposed on an outer surface of the distal end of the sheath catheter, and a passageway extending between the first and second sheath openings. The aspiration catheter is configured to pass through the first and second sheath openings and the passageway of the sheath catheter. The aspiration catheter includes a second balloon, a plurality of infusion ports, and an aspiration port disposed on an outer surface of a distal portion of the aspiration catheter. A relative position between the sheath catheter and the aspiration catheter controls the number of unblocked infusion ports.

In a second embodiment, the catheter apparatus comprises a sheath catheter combined with an aspiration catheter. The sheath catheter includes a first sheath opening in a proximal end of the sheath catheter, a second sheath opening in a distal end of the sheath catheter, a first balloon disposed on an outer surface of the distal end of the sheath catheter, and a passageway extending between the first and second sheath openings. The aspiration catheter is configured to pass through the first and second sheath openings and the passageway of the sheath catheter. The aspiration catheter includes a second balloon, a plurality of infusion ports, and an aspiration port disposed on an outer surface of a distal portion of the aspiration catheter. The sheath catheter includes a cover configured to block or unblock at least one of the plurality of infusion ports.

In another aspect of the present invention, there is provided a method for removing thrombosis using the disclosed catheter apparatus. The method includes: providing a sheath catheter comprising a first sheath opening in a proximal end of the sheath catheter, a second sheath opening in a distal end of the sheath catheter, a first balloon disposed on an outer surface of the distal end of the sheath catheter, and a passageway extending between the first and second sheath openings passing an aspiration catheter through the first and second sheath openings and the passageway of the sheath catheter, the aspiration catheter comprising a second balloon, a plurality of infusion ports, and an aspiration port disposed on an outer surface of a distal portion of the aspiration catheter; inserting a wire guide through a side opening of the aspiration catheter to a vessel having a thrombosis section; passing the distal portion of the aspiration catheter to the thrombosis section so that the second balloon passes by a distal end of the thrombosis section; advancing the sheath catheter until the first balloon are proximal to a proximal end of the thrombosis section; and manipulating a relative position between the sheath catheter and the aspiration catheter that controls the number of unblocked infusion ports.

### BRIEF DESCRIPTION OF THE DRAWINGS

The disclosure can be better understood with reference to the following drawings and description. The components in the figures are not necessarily to scale, emphasis instead being placed upon illustrating the principles of the disclosure. Moreover, in the figures, like referenced numerals designate corresponding parts throughout the different views.

FIG. 1 illustrates a first embodiment of the catheter apparatus;

FIG. 2 illustrates a distal end of the catheter apparatus of FIG. 1;

FIG. 3 illustrates a second embodiment the disclosed catheter apparatus;

FIG. 4 illustrates a first step for using the disclosed catheter apparatus;

FIG. 5 illustrates a second step for using the disclosed catheter apparatus;

FIG. 6 illustrates a third step for using the disclosed catheter apparatus;

FIG. 7 illustrates a fourth step for using the disclosed catheter apparatus; and

FIG. 8 is a block diagram of a method for removing the thrombosis from a blood vessel.

### DETAILED DESCRIPTION OF THE PREFERRED EMBODIMENTS

In the present application, the term “proximal” refers to a direction that is generally toward a physician during a medical procedure, while the term “distal” refers to a direction that is generally toward a target site within a patient's anatomy during a medical procedure.

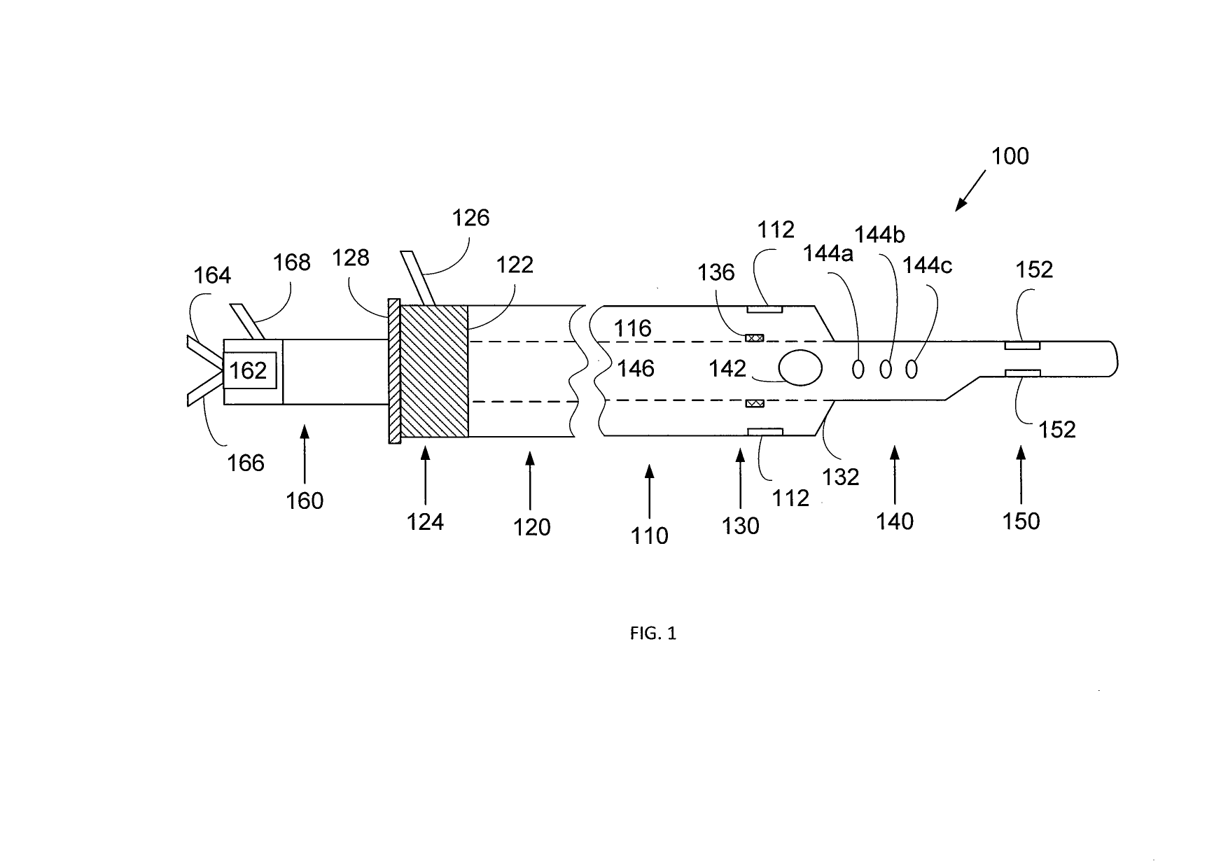

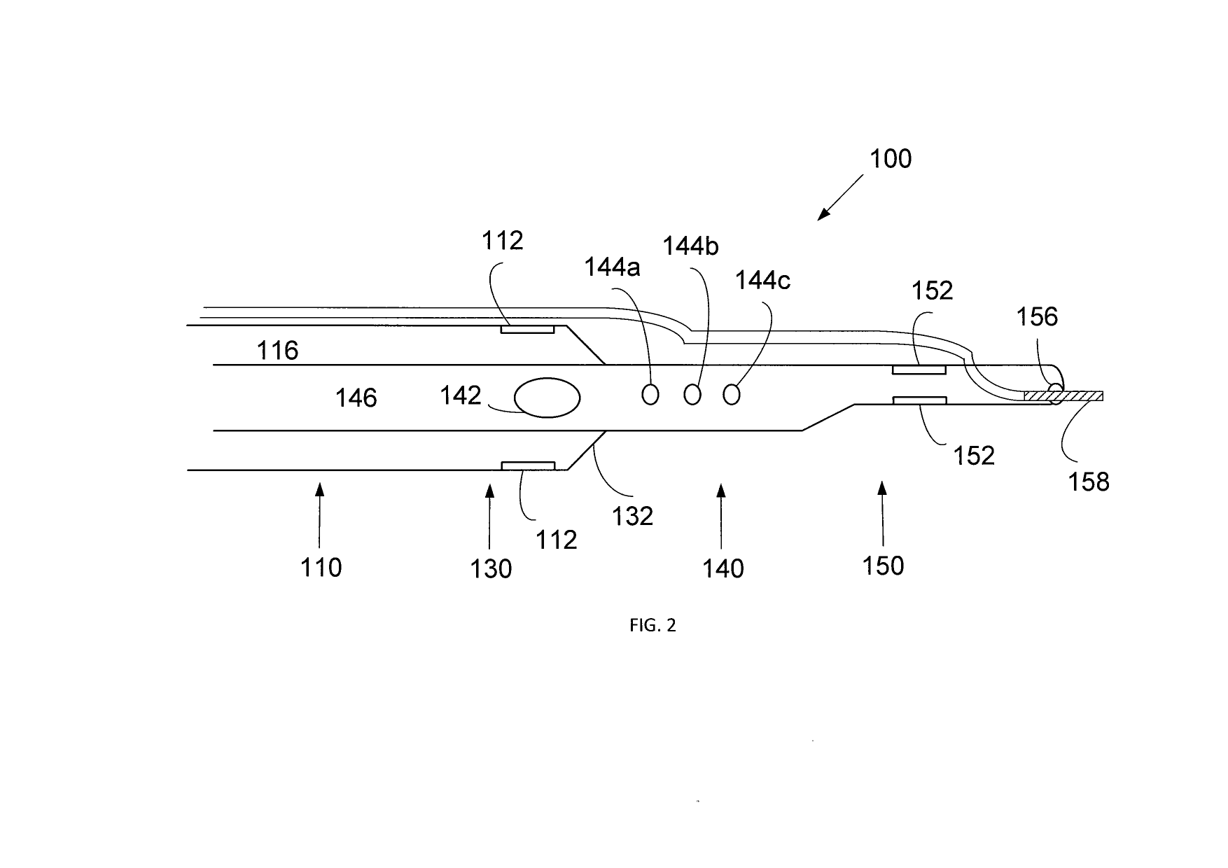

Referring now to FIGS. 1 and 2, a first embodiment of the catheter apparatus 100 is disclosed. The catheter apparatus 100 includes a sheath catheter 110 combined with an aspiration catheter 140. The catheter apparatus 100 may be used to remove deep vessel thrombosis.

The sheath catheter 110 includes a proximal end 120 and a distal end 130. There is a first sheath opening 122 in the proximal end 120 of the sheath catheter 110. There is a second sheath opening 132 in a distal end 130 of the sheath catheter. The sheath catheter 110 further includes a first balloon 112 disposed on an outer surface of the distal end 130 of the sheath catheter 110. There is a passageway 116 extending between the first sheath opening 122 and the second sheath opening 132. The sheath openings 122 and 132 may be configured to fit the aspiration catheter 140 and does not allow small broken clot fragment to get into the passageway 116. The sheath openings 122 and 132 may further be configured such that the aspiration catheter may be moved relatively to the sheath catheter. The sheath catheter 110 includes a hub 124 having a check flow valve 128 in the proximal end 120. The hub 124 also includes a proximal infusion port 126 configured to be connected with infusion devices that inflate or deflate the first balloon 112.

The aspiration catheter 140 is configured to pass through the first and second sheath openings 122 and 132 and the passageway 116 of the sheath catheter 110. The aspiration catheter 140 includes a second balloon 152 in a distal portion 150 of the aspiration catheter 140. There are also a plurality of infusion ports 144a, 144b, and 144c, and an aspiration port 142 disposed on an outer surface of the distal portion 150 of the aspiration catheter 140. The plurality of infusion ports 144a, 144b, and 144c are configured to communicate with proximal infusion source through a lumen 146 to infuse drug agents. The aspiration port 142 is configured to communicate with proximal aspiration sources through the same lumen 146 or a different passageway in the lumen 146 to such broken clots. The first and second balloons 112 and 152 are configured to isolate thrombosis in a vessel. The first and second balloons 112 and 152 may be replaced with any sealed expandable members.

A user may manipulate a relative position between the sheath catheter 110 and the aspiration catheter 140 by advancing or retracting the aspiration catheter 140 through a passageway 116 of the sheath catheter 110. The relative position between the sheath catheter 110 and the aspiration catheter 140 controls the number of unblocked infusion ports. Thus, a user can adjust the infusion rate of thrombolytics by manipulating the relative position of the sheath catheter 110 to the aspiration catheter 140. If the size of clot is relatively large, the user can manipulate the relative position so that more infusion ports are unblocked. If the size of clot is relatively small, the user can manipulate the relative position so that fewer infusion ports are unblocked. The relative position between the sheath catheter 110 and the aspiration catheter 140 also controls whether the aspiration port 142 is unblocked during the procedure. Alternatively or additionally, the relative position between the sheath catheter 110 and the aspiration catheter 140 may manipulate a variable length of the isolated thrombosis in the vessel.

The aspiration port 142 may have a larger opening compared with the infusion ports 144a, 144b, and 144c. The larger opening helps to remove the broken clot fragments more efficiently and reduce the duration of the procedure. Likewise, the larger opening may also help the remaining thrombolytics to be removed from the blood vessel promptly without additional delay. The aspiration catheter 140 may further include a bumper sleeve 136 to prevent back flow of fluid when the aspiration port 142 is unblocked in the blood vessel. For example, the bumper sleeve 136 may be a ring or a seal made of rubber or any similar material. Those skilled in the art would appreciate that the bumper sleeve 136 may also be a raised portion of the aspiration catheter 140 as long as it can prevent back flow of fluid to the passageway 116.

In FIG. 1, the aspiration catheter 140 has a distal portion 150 and a proximal portion 160. The proximal portion 160 includes a Y fitting 162 having a proximal aspiration port 164 and a proximal infusion port 166. The proximal aspiration port 164 is configured to aspirate the broken clot fragments through a lumen 146 extended through the aspiration catheter 140. The proximal aspiration port 164 communicates with the aspiration port 142 through the lumen 146. The proximal infusion port 166 is configured to infuse the drug agent such as thrombolytics through the same lumen 146 or a different passageway in the lumen 146. The proximal infusion port 166 communicates with the plurality of infusion ports 144a, 144b, and 144c through the same lumen 146 or a different passageway in the lumen 146. The proximal portion 160 of the aspiration catheter 140 also has a distal balloon inflation port 168 that is configured to be connected with infusion devices that inflate the second balloon 152 in the distal portion 150. As illustrated in FIG. 2, the distal portion 150 may include a side opening 156 configured to pass a wire guide 158 that guides the catheter apparatus 100 to the desire location in a blood vessel.

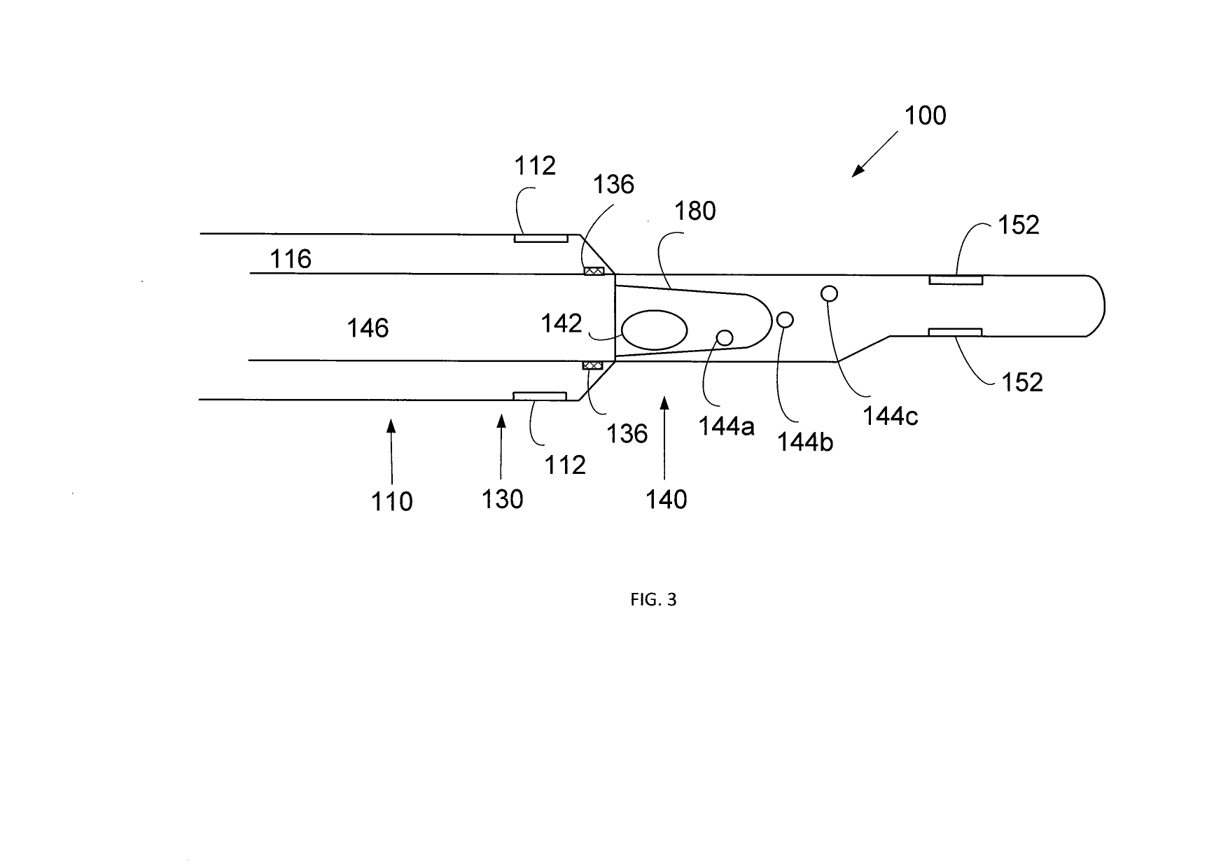

FIG. 3 illustrates a second embodiment the disclosed catheter apparatus 100. In this embodiment, the catheter apparatus 100 has a similar structure that includes a sheath catheter 110 combined with an aspiration catheter 140. Compared with the first embodiment, the sheath catheter 110 includes a cover 180 at the distal end 130. The cover 180 is configured to block the aspiration port 142. The cover 180 may be manipulated to unblock the aspiration port 142 without pulling the sheath catheter 110. For example, the sheath catheter 110 including the cover 180 may be rotated so that the cover 180 does not block the aspiration port 142. Additionally or alternatively, the cover 180 may further be configured to block or unblock at least one of the plurality of infusion ports 144a, 144b, or 144c. In this embodiment, the geometrical centers of the aspiration port 142 and the plurality of infusion ports 144a, 144b, or 144c may not be disposed in the same line as illustrated in FIG. 3. By adding this cover 180, the number of unblocked infusion ports is not necessarily determined by the isolated vessel length between the first balloon 112 and the second balloon 152. The user may have more control over the infusion rate.

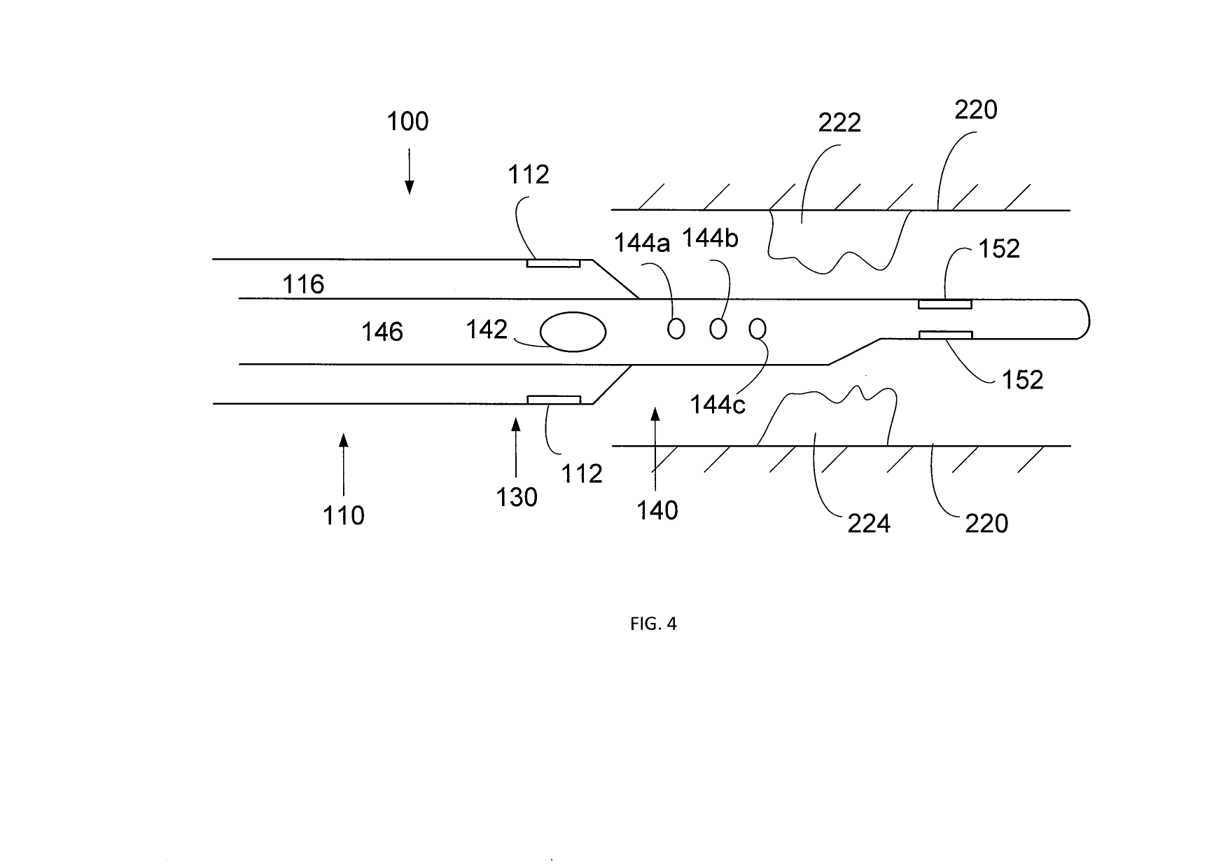

FIG. 4 illustrates a first step for using the disclosed catheter apparatus. In this step, the user positions the catheter apparatus 100 within the vessel section 220 having thrombosis 222 and 224 on the vessel wall. The user may use ultrasound imaging or other medical imaging methods to determine sizes and locations of the thrombosis 222 and 224. The user may also use marker bands on the catheter apparatus 100 to position the catheter apparatus 100 at the desired vessel with the help of these medical imaging methods. The user may use a wire guide 158 illustrated in FIG. 2 in this step.

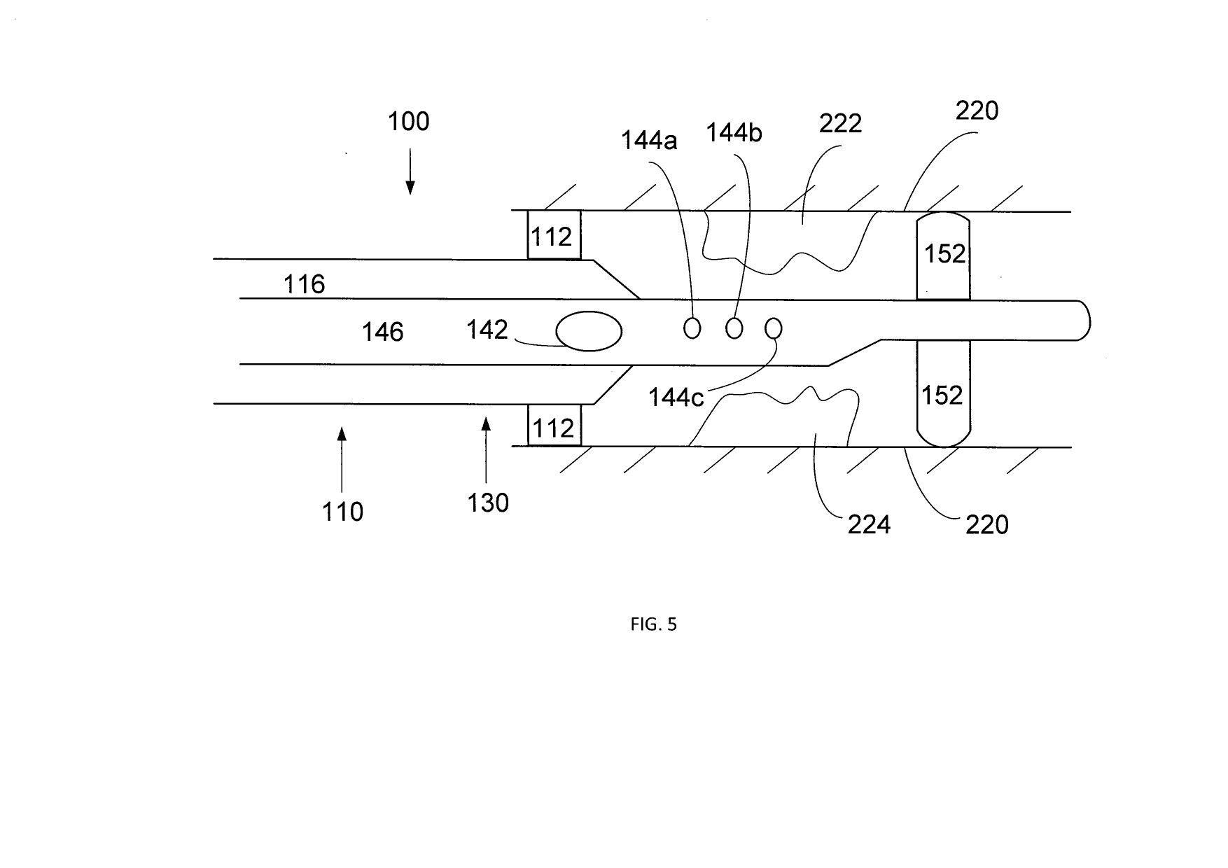

FIG. 5 illustrates a second step for using the disclosed catheter apparatus. In this step, the user first inflates the second balloon 152 in the distal end of the aspiration catheter 140 at a location distal to the thrombosis 222 and 224. The user then adjusts the relative position between the sheath catheter 110 and the aspiration catheter 140 according to the size and condition of the thrombosis 222 and 224 so as to position the first balloon 112 proximal of thrombosis 222 and 224. After that, the user inflates the first balloon 112 disposed in the distal end 130 of the sheath catheter 110. When the balloons 112 and 152 are inflated, the thrombosis 222 and 224 are isolated between the first and second balloons 112 and 152. Once the thrombosis 222 and 224 are isolated, the user may infuse drug agents such as thrombolytics to the isolated thrombosis 222 and 224 through the infusion ports 144a, 144b, and 144c. The catheter apparatus may have additional ports downstream of the second balloon 152 to provide perfusion downstream of the thrombosis 222 and 224 being treated.

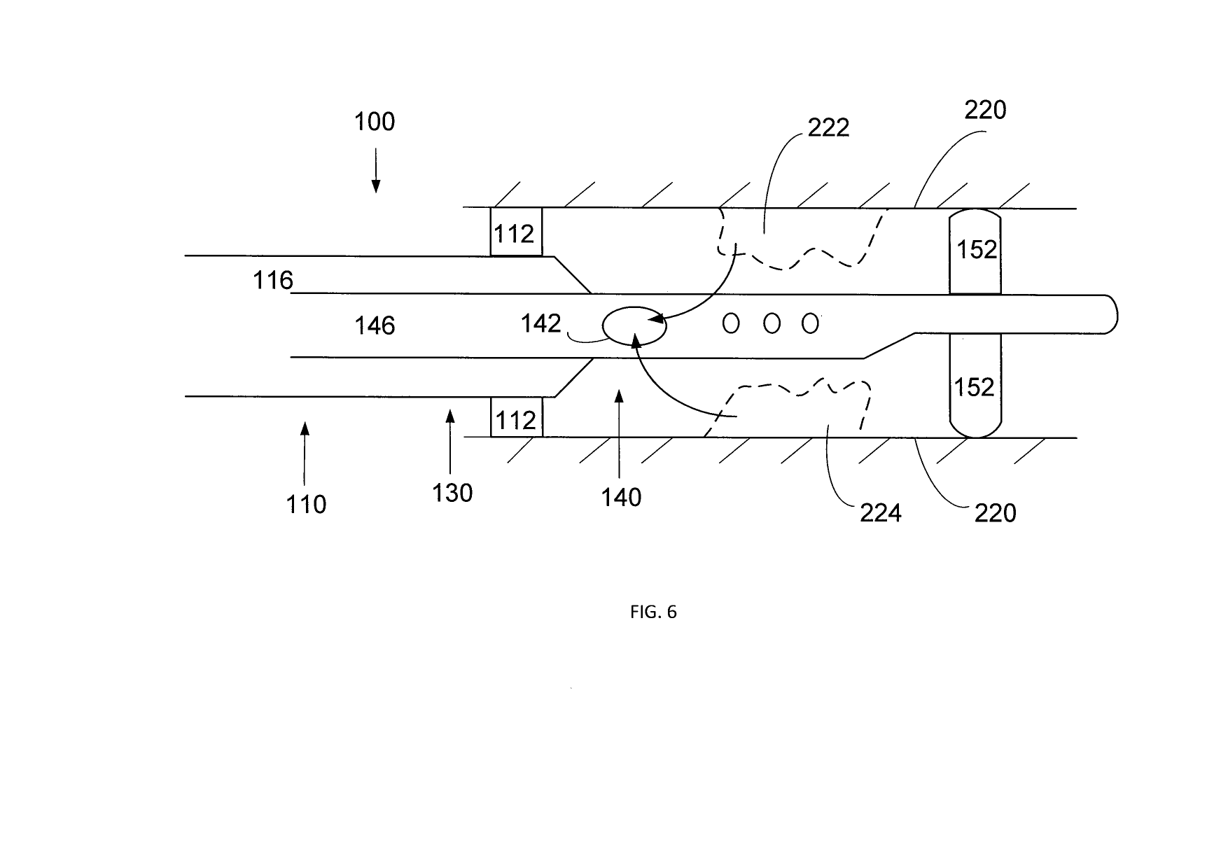

FIG. 6 illustrates a third step for using the disclosed catheter apparatus to treat the thrombosis 222 and 224. After the drug agents have worked for a predetermined period of time, the thrombosis 222 and 224 would be broken into clot fragments. The user then manipulates the relative position between the sheath catheter 110 and the aspiration catheter 140 to unblock the aspiration port 142. For example, the user may pull the sheath catheter 110 in the first embodiment or release the cover 180 in the second embodiment to unblock the aspiration port 142. Once the aspiration port 142 is unblocked in the vessel, the user may remove the broken clot fragments through the unblocked aspiration port 142. During the period of treating and removing the broken clot fragments, the treated vessel section is kept isolated by the inflated balloons. Thus, the broken clot fragments would not be move to undesired location. Also, most of the remaining drug agents would be removed without affecting other parts of the vessel. This step may be repeated by reclosing the aspiration port and infuse more drug agents if necessary.

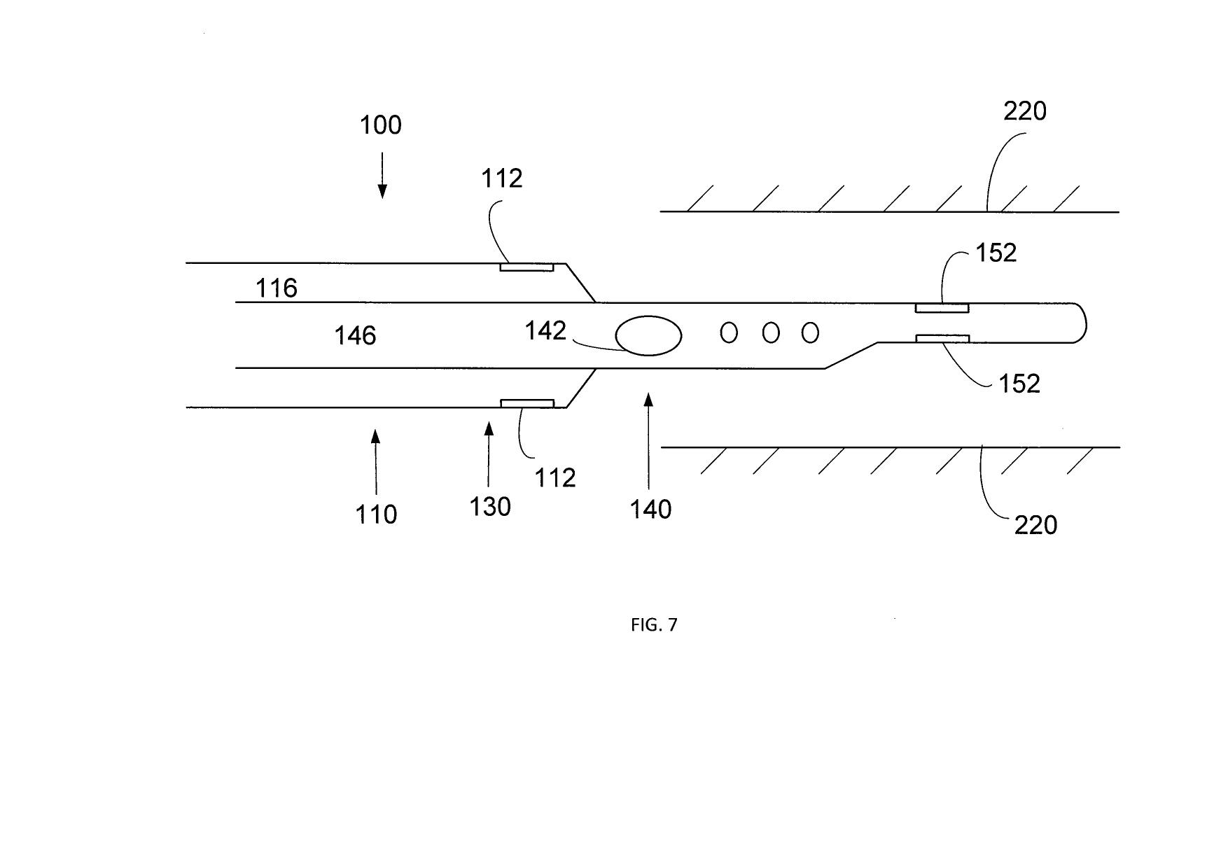

FIG. 7 illustrates a fourth step for using the disclosed catheter apparatus. After the broken clot fragments and the drug agents are removed from the treated vessel section 220, the user then deflates the balloons 112 and 152 and removes the catheter apparatus 100 from the treated vessel section 220.

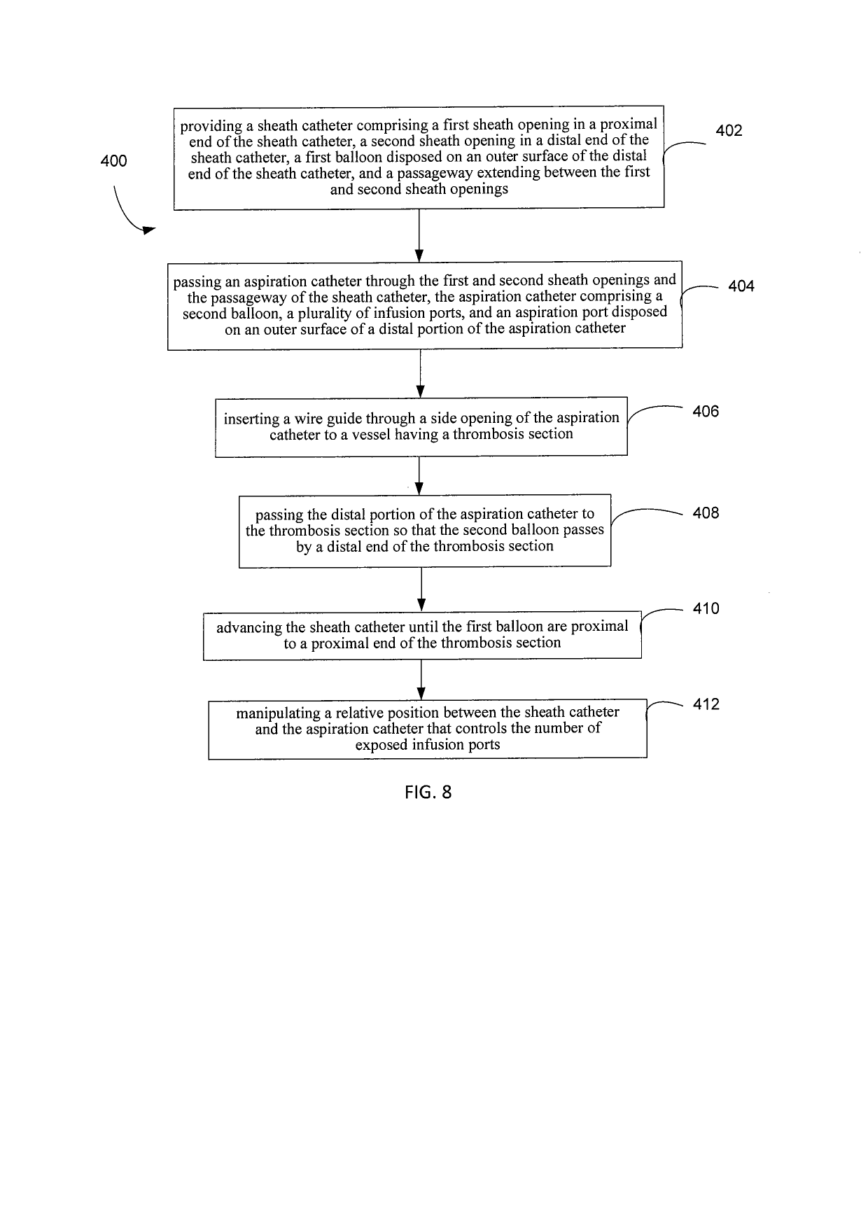

FIG. 8 is a block diagram of a method 400 for removing the thrombosis from a blood vessel using the disclosed catheter apparatus 100. The method 400 comprises the following steps. In step 402, the user provides a sheath catheter comprising a first sheath opening in a proximal end of the sheath catheter, a second sheath opening in a distal end of the sheath catheter, a first balloon disposed on an outer surface of the distal end of the sheath catheter, and a passageway extending between the first and second sheath openings.

In step 404, the user passes an aspiration catheter through the first and second sheath openings and the passageway of the sheath catheter. The aspiration catheter includes a second balloon, a plurality of infusion ports, and an aspiration port disposed on an outer surface of a distal portion of the aspiration catheter.

In step 406, the user inserts a wire guide through a side opening of the aspiration catheter to a vessel having a thrombosis section. In step 408, the user passes the distal portion of the aspiration catheter to the thrombosis section so that the second balloon passes by a distal end of the thrombosis section. In step 410, the user advances the sheathe catheter until the first balloon are proximal to a proximal end of the thrombosis section. The user may then expand the first balloon after expanding the second balloon to isolate the thrombosis section to be treated.

Once the thrombosis section is isolated, in step 412, the user manipulates a relative position between the sheath catheter and the aspiration catheter that controls the number of unblocked infusion ports. The user delivers drug agents though the unblocked infusion ports to the isolated thrombosis section. Then the user waits for the drug agents to work on the thrombosis section for a predetermined period of time. After the thrombosis are broken to clot fragments, the user then manipulates the relative position between the sheath catheter and the aspiration catheter to unblock the aspiration port. The user may then remove the broken clot fragments through the unblocked aspiration port. If necessary, the steps may be repeated to infuse more drug agents to the isolated vessel section and aspirate the broken clot fragments afterwards. After the whole procedure is finished, the user may deflate the balloons and remove the catheter apparatus from the vessel.

Although the illustrated catheter apparatus in FIGS. 1-7 includes a proximal balloon 112 and a distal balloon 152, those skilled in the art will appreciate that the catheter apparatus may include multiple proximal balloons and distal balloons as long as the expanded proximal and distal balloons isolate the thrombosis.

The disclosed catheter apparatus and corresponding method provides a user such as a physician to use one single device instead of two separate devices. The disclosed catheter apparatus isolates the vessel section to be treated and uses the drug agents more efficiently. The user may manipulate the length of the isolated vessel section. The user may also manipulate the number of unblocked infusion ports and thus control the infusion rate. Additionally, a relatively large aspiration port helps to remove the broken clot fragments faster.

While various embodiments of the invention have been described, it will be apparent to those of ordinary skill in the art that many more embodiments and implementations are possible within the scope of the invention. The different aspects of the described embodiments may be combined together to improve the performance of the hemodialysis catheter. Accordingly, the invention is not to be restricted except in light of the attached claims and their equivalents. Moreover, the advantages described herein are not necessarily the only advantages of the invention and it is not necessarily expected that every embodiment of the invention will achieve all of the advantaged described.

What is claimed is:

1. An apparatus for removing thrombosis, the apparatus comprising:
a sheath catheter comprising a first sheath opening in a proximal end of the sheath catheter, a second sheath opening in a distal end of the sheath catheter, a first balloon disposed on an outer surface of the distal end of the sheath catheter, and a passageway extending between the first and second sheath openings, and
an aspiration catheter configured to pass through the first and second sheath openings and the passageway of the sheath catheter, the aspiration catheter comprising a second balloon, a plurality of infusion ports, and an aspiration port disposed on an outer surface of a distal portion of the aspiration catheter,
wherein a relative position between the sheath catheter and the aspiration catheter controls the number of unblocked infusion ports.

2. The apparatus of claim 1, wherein the aspiration catheter comprises a Y fitting having a proximal aspiration port and a proximal infusion port in a proximal portion of the aspiration catheter.

3. The apparatus of claim 1, wherein the relative position between the sheath catheter and the aspiration catheter controls whether the aspiration port is unblocked.

4. The apparatus of claim 1, wherein the aspiration catheter comprises a side opening configured to pass a wire guide therein.

5. The apparatus of claim 1, wherein the first and second balloons are configured to isolate thrombosis in a vessel.

6. The apparatus of claim 5, wherein the relative position between the sheath catheter and the aspiration catheter manipulate a variable length of the isolated thrombosis in the vessel.

7. The apparatus of claim 6, wherein the distal portion of the aspiration catheter is tapered that allows the distal portion to pass through the thrombosis.

8. The apparatus of claim 1, wherein the sheath catheter comprises a hub having a check flow valve in the proximal end of the sheath catheter, and, wherein the hub further comprises a first balloon inflation port configured to inflate the first balloon.

9. The apparatus of claim 2, wherein the proximal aspiration port and the proximal infusion port respectively communicates with the plurality of infusion ports and the aspiration port through a lumen in the aspiration catheter.

10. The apparatus of claim 1, wherein the aspiration catheter comprises a second balloon inflation port configured to inflate the second balloon.

11. The apparatus of claim 1, wherein the distal portion of the aspiration catheter comprises bumper sleeve configured to prevent back flow of fluid.

12. The apparatus of claim 5, wherein the aspiration catheter is configured to deliver drug agents to the isolated thrombosis in the vessel through the plurality of infusion ports.

13. A method for removing thrombus, the method comprising:
providing a sheath catheter comprising a first sheath opening in a proximal end of the sheath catheter, a second sheath opening in a distal end of the sheath catheter, a first balloon disposed on an outer surface of the distal end of the sheath catheter, and a passageway extending between the first and second sheath openings;
passing an aspiration catheter through the first and second sheath openings and the passageway of the sheath catheter, the aspiration catheter comprising a second balloon, a plurality of infusion ports, and an aspiration port disposed on an outer surface of a distal portion of the aspiration catheter;
inserting a wire guide through a side opening of the aspiration catheter to a vessel having a thrombosis section;
passing the distal portion of the aspiration catheter to the thrombosis section so that the second balloon passes by a distal end of the thrombosis section;
advancing the sheath catheter until the first balloon are proximal to a proximal end of the thrombosis section; and
manipulating a relative position between the sheath catheter and the aspiration catheter that controls the number of unblocked infusion ports.

14. The method of claim 13, further comprising expanding the first balloon after expanding the second balloon to isolate the thrombosis section.

15. The method of claim 14, further comprising delivering drug agents though the unblocked infusion ports to the isolated thrombosis section.

16. The method of claim 15, further comprising manipulating the sheath catheter and exposing the aspiration port after breaking the thrombosis.

17. The method of claim 16, further comprising removing the broken thrombosis through the unblocked aspiration port.

18. An apparatus for removing thrombus, the apparatus comprising:
a sheath catheter comprising a first sheath opening in a proximal end of the sheath catheter, a second sheath opening in a distal end of the sheath catheter, a first balloon disposed on an outer surface of the distal end of the sheath catheter, and a passageway extending between the first and second sheath openings, and
an aspiration catheter configured to pass through the first and second sheath openings and the passageway of the sheath catheter, the aspiration catheter comprising a second balloon, a plurality of infusion ports, and an aspiration port disposed on an outer surface of a distal portion of the aspiration catheter,
wherein the sheath catheter further comprises a cover configured to block or unblock at least one of the plurality of infusion ports.

19. The apparatus of claim 18, wherein the cover is configured to block or unblock the aspiration port.

20. The apparatus of claim 18, wherein the distal portion of the aspiration catheter comprises bumper sleeve configured to prevent back flow of fluid.
